(19) United States
(12) Patent Application Publication
(10) Pub. No.: US 2026/0174818 A1
(43) Pub. Date: Jun. 25, 2026

(54) PEPTIDE ACTIVATING VITAMIN D RECEPTOR, AND COSMETIC COMPOSITION CONTAINING SAME

(71) Applicant: SUPADELIXIR INC., Chuncheon-si (KR)

(72) Inventors: Jang Hee HAHN, Chuncheon-si (KR); Min Seo KIM, Chuncheon-si (KR)

(21) Appl. No.: 18/834,596

(22) PCT Filed: Sep. 12, 2023
(86) PCT No.: PCT/KR2023/013668

§ 371 Date: Jul. 30, 2024

(30) Foreign Application Priority Data
Sep. 12, 2023 (KR) 10-2023-0121127

(51) Int. Cl. A61K 38/00; A61Q 17/04; A61Q 19/00; A61Q 19/08

(52) U.S. Cl. CPC A61K 38/00; A61Q 17/04; A61Q 19/00; A61Q 19/08

(57) ABSTRACT

Proposed are a peptide activating vitamin D receptors and a cosmetic composition using the same.

### TECHNICAL FIELD

The present disclosure relates to a peptide activating vitamin D receptors and a cosmetic composition using the same.

### BACKGROUND ART

Vitamin D is an important hormone for bone growth and bone maintenance and mineral homeostasis. Vitamin D binds to a vitamin D receptor (VDR) in various tissues and cells in the human body and regulates the expression of genes involved in cell growth and differentiation and immune function. Meanwhile, research has been conducted to solve skin troubles (hair loss, inflammation, and acne) and to improve skin conditions (skin rejuvenation) related to vitamin D and vitamin D receptors.

Therefore, as shown in the patent document below, activators are being developed to activate vitamin D receptors.

### Patent Document

Korean Patent Application Publication No. 10-2010-0099181 (published on Sep. 10, 2010), titled “New Vitamin D Receptor Activators and Methods of Making”

However, conventional vitamin D receptor activators have limitations in that the vitamin D receptor activators are not effective enough or may cause side effects when used since the activators are mostly chemically synthesized.

### DISCLOSURE

### Technical Problem

The present disclosure was devised to solve the problems.

The objective of the present disclosure is to provide a peptide activating vitamin D receptors and a cosmetic composition using the same, both capable of being used to alleviate acne, hair loss, and inflammation, or to rejuvenate skin.

### Technical Solution

To achieve the described objective, the present disclosure is implemented by embodiments having the following configuration.

According to one embodiment of the present disclosure, a peptide according to the present disclosure is made of any one amino acid sequence selected from ones represented by SEQ ID NOs: 1 to 3.

According to another embodiment of the present disclosure, the peptide according to the present disclosure activates vitamin D receptors.

According to yet another embodiment of the present disclosure, the peptide according to the present disclosure is used to alleviate skin troubles or to rejuvenate skin.

According t another embodiment of the present disclosure, the peptide according to the present disclosure is used to alleviate atopy, acne, hair loss, and inflammation or to rejuvenate skin.

According to yet another embodiment of the present disclosure, a cosmetic composition according to the present disclosure contains a peptide including any one amino acid sequence selected from ones represented by SEQ ID NOs: 1 to 3.

According to yet another embodiment of the present disclosure, a composition to alleviate skin troubles or to rejuvenate skin according to the present disclosure contains a peptide including any one amino acid sequence selected from ones represented by SEQ ID NOs: 1 to 3.

According to yet another embodiment of the present disclosure, the cosmetic composition according to the present disclosure is used to alleviate atopy, acne, hair loss, and inflammation or to rejuvenate skin.

### Advantageous Effects

The following effects can be achieved by the described embodiments of the present disclosure.

By activating vitamin D receptors using a peptide including any one amino acid sequence selected from ones represented by SEQ ID NOs: 1 to 3, the present disclosure can be effective in alleviating acne, hair loss, and inflammation or rejuvenating skin.

### DESCRIPTION OF DRAWINGS

FIG. 1 is a graph showing the effects of peptides on the physical binding between VDR and RXR according to one embodiment of the present disclosure;

FIG. 2 is a graph showing the effects of peptides on the intracellular expression of S100A3 according to another embodiment of the present disclosure;

FIG. 3 is a graph showing the effects of peptides on inhibiting the increase in LPS-induced NFkB nuclear migration according to yet another embodiment of the present disclosure; and

FIG. 4 is a graph showing the effects of peptides on increasing the transcriptional activity of P53 according to yet another embodiment of the present disclosure.

### BEST MODE

Hereinafter, peptides activating vitamin D receptors and cosmetic compositions containing the same according to the present disclosure will be described in detail with reference to the attached drawings. Unless otherwise specified, all terms in this specification have the same general meaning as understood by those skilled in the art to which the present disclosure pertains. When there is a conflict with the meaning of the terms used herein, this specification follows the definitions used in the specification. Additionally, detailed descriptions of well-known functions and configurations that may unnecessarily obscure the gist of the present disclosure are omitted. Throughout the specification, when a part “includes” a certain component, this means that the part may further include other components rather than excluding other components unless specifically stated to the contrary.

According to one embodiment, the present disclosure relates to peptides activating vitamin D receptors. The peptides are made of an amino acid sequence of SEQ ID NO: 1 (Glu-Val-Phe-Gly), SEQ ID NO: 2 (Val-Ile-Gly-Phe), or SEQ ID NO: 3 (Ile-Gly-Phe-Ala). The peptides may alleviate skin troubles and improve skin conditions by activating vitamin D receptors. Specifically, the peptides prevent excessive secretion of sebum and provide anti-inflammatory effects, thereby improving inflammatory skin conditions through the alleviation of acne and atopy, preserving hair or preventing hair loss, and promoting skin rejuvenation. The peptides may be used in cosmetic compositions and therapeutic compositions.

According to another embodiment, the present disclosure relates to cosmetic compositions containing a peptide including any one amino acid sequence selected from ones represented by SEQ ID NOs: 1 to 3. The cosmetic compositions may alleviate skin troubles such as acne, hair loss, and skin inflammation, and rejuvenate skin. Various known ingredients that may provide formulation stability and additional skin condition improvement effects may be further included in the cosmetic compositions.

Hereinafter, the present disclosure will be described in more detail through examples. However, these are only for explaining the present disclosure in more detail, and the scope of the present disclosure is not limited thereto.

### <Example 1> Synthesis of Peptides

Peptides represented by SEQ ID NOs: 1 to 3 and listed in Table 1 below were synthesized by an FMOC solid-phase method using an automated synthesizer (PeptrEx-R48, Peptron, Korea). The synthesized peptides were purified and analyzed by reverse-phase high-performance liquid chromatography (Prominence LC-20AB, Shimadzu, Japan) using a C18 analytical RP column (Shiseido Capcell Pak). The synthesized peptides were identified by mass spectrometry (HP 1100 Series LC/MSD, Hewlett-Packard, USA).

TABLE 1

| Peptide name | Sequence number | Amino acid sequence |
|---|---|---|
| VDR Pep 1 | Sequence number 1 | Glu-Val-Phe-Gly |
| VDR Pep 2 | Sequence number 2 | Val-Ile-Gly-Phe |
| VDR Pep 3 | Sequence number 3 | Ile-Gly-Phe-Ala |

### <Example 2> Confirmation of Whether Peptides of Example 1 Activate Vitamin D Receptors and Increase Physical Binding Between VDR and RXR

1. Vitamin D receptors (VDRs) bind to Retinoid X Receptors (RXRs) to regulate transcription of target DNA. To confirm whether the peptides of Example 1 activate VDRs, the effects of the peptides on the physical binding between VDR and RXR were evaluated using an in-situ proximity ligation assay (PLA) method.

2. Specifically, 4.5×10⁴ HaCaT cells (human keratinocytes) were seeded into each well of a 24-well microplate with DMEM serum medium. The cells were cultured in an incubator at a temperature of 37° C. and with 5% CO₂ for 24 hours. Next, the cells were treated with each of the peptides (VDR Pep 1 to 3) of Example 1, and the cells were incubated under the same conditions for 30 minutes. The concentration of each peptide in the medium was set to 1 and 10 μM. The control group received no treatment. Afterward, the cells in each well were washed with PBS and fixed by treatment with 2% formaldehyde for 15 minutes, and then the cells were treated with 0.1% TritonX-100 for 5 minutes to increase antibody permeability into the cells. Anti-VDR monoclonal antibodies (Santa Cruz, USA) and anti-RXR polyclonal antibodies (Santa Cruz, USA) were added, and PLA probes were applied using an in-situ PLA kit (Sigma-Aldrich). Subsequently, hybridization, ligation, amplification, and mounting steps were performed.

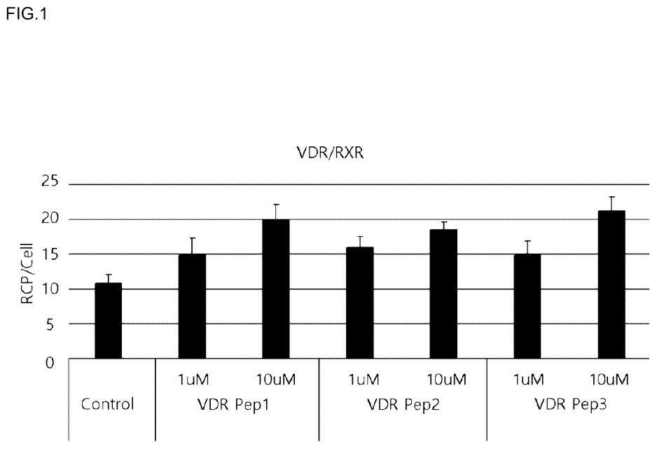

3. Afterward, the physical interaction of VDR and RXR antibodies was quantified by measuring luminescence signals (PLA signals) detected in each cell using a confocal laser microscope (Olympus Fluoview FW1000; Olympus, Tokyo, Japan), and the results are shown in FIG. 1.

4. As seen in FIG. 1, when treated with VDR Pep 1 to 3 of Example 1, the physical binding between VDRs and RXRs increased compared to the untreated control group. That is, VDR Pep 1 to 3 activated VDR, thereby increasing the physical binding between VDR and RXR, and ultimately alleviating skin diseases (hair loss, skin inflammation, and acne) and promoting skin rejuvenation.

### <Example 3> Evaluation of Whether Peptides of Example 1 have Effects on Intracellular Expression Amount of S100A3, Ca²⁺ Binding Protein that Promotes Hair Differentiation

1. To confirm the effects of the peptides of Example 1 on preventing hair loss, the effects of the peptides on the expression of S100A3, which was a Ca²⁺ binding protein that promoted hair differentiation, were confirmed by Western blotting technique.

2. Specifically, HaCaT cells were prepared to have a concentration of 5×10⁶ per well and then treated with each of the peptides of Example 1 (VDR Pep 1 to 3). The cells were incubated under the same conditions for 24 hours. The concentration of each peptide in the medium was set to 1 and 10 μM. The control group received no treatment. The HaCaT cells were washed three times with PBS. Next, the HaCaT cells were lysed using a 1% NP40 lysis buffer (1% Nonidet P40, 0.1 M NaCl, 0.05 M Tris (pH 8.0), 5 mM EDTA) containing 0.1 μM phenylmethylsulfonyl fluoride (PMSF), 1 μg/ml pepstatin A, 10 μg/ml leupeptin, 1 μg/ml aprotinin, and 1 mM Na₃VO₄.

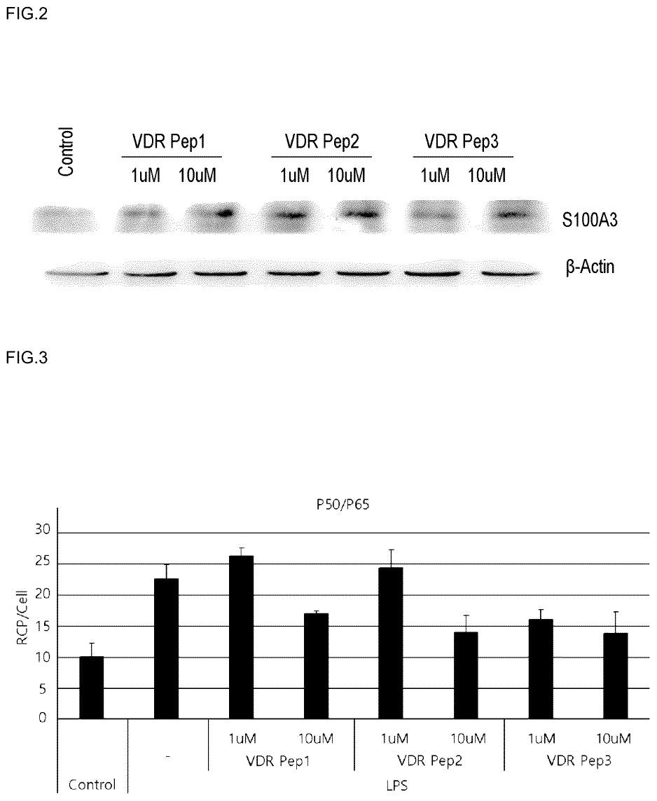

3. Cell lysates were quantified using a Brad ford assay, and then samples were prepared for electrophoresis. Immunoprecipitates of the samples were subjected to electrophoresis on a 15% polyacrylamide gel. After transferring the unfolded proteins to a nitrocellulose membrane, the proteins were treated with a blocking solution (Tris-buffered saline (TBS) containing 0.05% Tween 20 and 3% bovine serum albumin) for about 1 hour at room temperature. Next, the proteins were subject to a reaction for 2 hours in TBS buffer containing anti-S100A3 polyclonal antibodies (Santa Cruz, CA, USA), and then washed with TBS buffer containing 0.05% Tween 20. The proteins were treated with horseradish peroxidase-conjugated anti-rabbit IgG (Santa Cruz, CA, USA) for 1 hour at room temperature, and then were washed five times with TBS containing 0.05% Tween 20. The proteins were developed using an antibody detection kit (Ab frontier, Korea), and the results are shown in FIG. 2. To confirm that the same amount of the cell lysates were subjected to electrophoresis, anti-beta actin monoclonal antibodies (Santa Cruz, CA, USA) were used, and then actins were recognized.

4. As seen in FIG. 2, when treated with VDR Pep 1 to 3 of Example 1, the expression of S100A3, which was a Ca²⁺ binding protein that promoted hair differentiation, was promoted. That is, VDR Pep 1 to 3 activated VDRs and alleviated hair loss.

### <Example 4> Evaluation of Whether Peptides of Example 1 have Inhibition Effect on Increase in LPS-Induced NFkB Nuclear Migration

1. When cells are stimulated with LPS, NFkB (P50 and P65) moves into the nucleus and initiates an inflammatory response. To confirm whether the peptides of Example 1 inhibit this, cells were treated with LPS and the peptides of Example 1, and then the degree of movement of P50 and P65 into the nucleus was evaluated using an in-situ proximity ligation assay (PLA) method.

2. Specifically, 4.5×10⁴ HaCaT cells (human keratinocytes) were seeded into each well of a 24-well microplate with DMEM serum medium. The cells were cultured in an incubator at a temperature of 37° C. and with 5% CO₂ for 24 hours. Next, the cells were treated with 10 μg/ml of LPS and each of the peptides (VDR Pep 1 to 3) of Example 1 in the medium, and the cells were incubated under the same conditions for 30 minutes. The concentration of each peptide in the medium was set to 1 and 10 μM. Out of two control groups, one received no treatment, and the other was treated with LPS alone. The cells in each well were washed with PBS and fixed by treatment with 2% formaldehyde for 15 minutes, and then the cells were treated with 0.1% TritonX-100 for 5 minutes to increase antibody permeability into the cells. Anti-P65 polyclonal antibodies (CST, USA) and anti-P50 monoclonal antibodies (Santa Cruz, CA, USA) were added, and PLA probes were applied using an in-situ PLA kit (Sigma-Aldrich). Subsequently, hybridization, ligation, amplification, and mounting steps were performed.

3. The interaction between P65 and P50 antibodies in the nucleus was quantified by measuring luminescence signals (PLA signals) detected in each cell using a confocal laser microscope (Olympus fluoview FW1000; Olympus, Tokyo, Japan), and the results are shown in FIG. 3.

4. As seen in FIG. 3, when treated with VDR Pep 1 to 3 of Example 1, the increase in nuclear translocation of NFkB induced by LPS stimulation was reduced compared to the untreated control groups. That is, VDR Pep 1 to 3 activated VDRs and alleviated inflammation.

### <Example 5> Evaluation of Whether Peptides of Example 1 Increase Transcriptional Activity of P53

1. To confirm whether the peptides of Example 1 increase the transcriptional activity of P53, which was related to cell anti-aging, cells were treated with the peptides of Example 1, and then the degree of binding between P53 and RNA Polymerase II (POLII) was confirmed using an in-situ proximity ligation assay (PLA) method.

2. Specifically, 4.5×10⁴ HaCaT cells (human keratinocytes) were seeded into each well of a 24-well microplate with DMEM serum medium. The cells were cultured in an incubator at a temperature of 37° C. and with 5% CO₂ for 24 hours. Next, the cells were treated with each of the peptides (VDR Pep 1 to 3) of Example 1 in the medium, and the cells were incubated under the same conditions for 30 minutes. The concentration of each peptide in the medium was set to 1 and 10 μM. The control group received no treatment. The cells in each well were washed with PBS and fixed by treatment with 2% formaldehyde for 15 minutes, and then the cells were treated with 0.1% TritonX-100 for 5 minutes to increase antibody permeability into the cells. Anti-P53 polyclonal antibodies (CST, USA) and anti-POLII monoclonal antibodies (Santa Cruz, CA, USA) were added, and PLA probes were applied using an in-situ PLA kit (Sigma-Aldrich). Subsequently, hybridization, ligation, amplification, and mounting steps were performed.

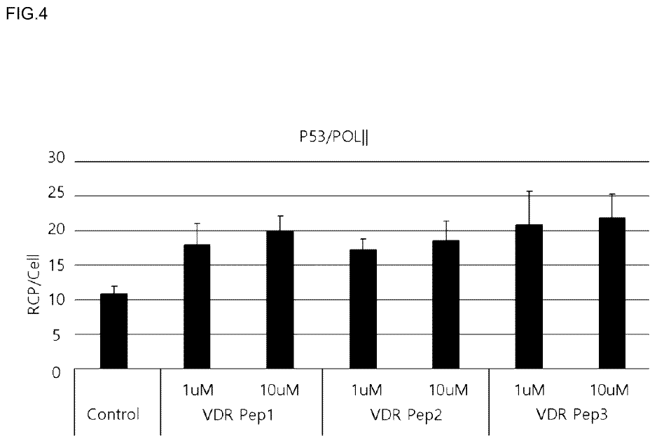

3. The interaction between P65 and P50 antibodies in the nucleus was quantified by measuring PLA signals detected in each cell using a fluorescence microscopy (Logosbio/CelenaS, Korea), and the results are shown in FIG. 4.

4. As seen in FIG. 4, when treated with VDR Pep 1 to 3 of Example 1, the binding of P53 to RNApolymeraseII increased compared to the untreated control group. That is VDR Pep 1 to 3 activated VDRs and increased the transcriptional activity of P53 to prevent cell aging.

### <Example 6> Confirmation of Whether Cosmetic Compositions Containing Peptides of Example 1 have Sebum Inhibition Effect Sebum Through Skin Patch Experiment

1. Cosmetic compositions were prepared by adding each peptide prepared in Example 1 to a commercially available solubilized ampoule base at a concentration of 50 ppm. Cosmetic composition 1 was a soluble ampoule base without the peptides of Example 1. Cosmetic composition 2 was one with VDR Pep 1. Cosmetic composition 3 was one with VDR Pep 2. Cosmetic composition 4 was one with VDR Pep 3.

2. Twenty-one male adults in their 40s with excessive sebum secretion were asked to wipe their forehead area with 70% ethanol and dry the area. Detail tape was attached to the forehead area and then removed to check. Afterward, the same amount of each of the cosmetic compositions 1 to 4 was applied onto the forehead area every morning and evening for 3 weeks (the experiment was conducted by soaking a sheet with each of the cosmetic compositions and attaching the sheet to the forehead area for a certain period). The next day after three weeks, the detailed tape attached to the forehead area was removed to check. A relative reduction in sebum was confirmed by checking the detailed tape before and after applying the cosmetic compositions.

3. The experiment showed that no irritation occurred when the skin of the forehead area was checked after applying the cosmetic compositions for 3 weeks. As a result of confirming the relative reduction in sebum before and after applying the cosmetic compositions, no significant reduction in sebum was confirmed when cosmetic composition 1, the control group, was applied. Meanwhile, a significant reduction in sebum was confirmed when cosmetic compositions 2 to 4 were applied. That is, VDR Pep 1 to 3 of Example 1 suppressed sebum production.

### <Example 7> Confirmation of Whether Cosmetic Compositions Containing Peptides of Example 1 have Anti-Inflammatory Effects Through Skin Patch Experiment

1. Twenty-one adults with inflammatory skin diseases such as atopy and acne were asked to apply the same amount of each of the cosmetic compositions 1 to 4 prepared in Example 6-1 to the affected skin area every morning and evening for 3 weeks. The next day after three weeks, each participant was asked to make a subjective evaluation of the improvement in skin condition, and the results are shown in Table 3. In the evaluation, the score was 5 for significant improvement, 4 for improvement, 3 for average, 2 for deterioration, and 1 for significant deterioration.

2. Looking at Table 2, cosmetic compositions 2 to 4 had significant skin improvement effects. That is, VDR Pep 1 to 3 might be used in cosmetic compositions to improve skin conditions by alleviating inflammatory skin diseases such as atopy and acne.

TABLE 2

| Cosmetic | | | | |
|---|---|---|---|---|
| composition | 1 | 2 | 3 | 4 |
| Skin | 2.6 | 4.4 | 4.6 | 4.7 |
| improvement | | | | |

### <Example 8> Confirmation of Whether Cosmetic Compositions Containing Peptides of Example 1 have Skin Rejuvenation Effects Through Skin Patch Experiment

1. Twenty-one elderly adults in their 60s were asked to apply the same amount of each of the cosmetic compositions 1 to 4 prepared in Example 6-1 to the face every morning and evening for 10 weeks. After the 10 weeks, each participant was asked to make a subjective evaluation of the skin rejuvenation effect, taking into account wrinkle reduction, skin elasticity, and scar reduction.

2. As a result of the experiment, the participants evaluated that there was no skin rejuvenation effect when using cosmetic composition 1, which was the control group. Meanwhile, the participants evaluated that when cosmetic compositions 2 to 4 were used, wrinkles were relatively reduced, skin elasticity improved, and a skin rejuvenation effect was recognized due to scar reduction.

### <Example 9> Confirmation of Whether Cosmetic Compositions Containing Peptides of Example 1 have Alleviation Effect on Hair Loss Through Skin Patch Experiment

1. Cosmetic compositions were prepared with the composition ratio shown in Table 2 below to confirm the effect of alleviating hair loss.

TABLE 3

| | |
|---|---|
| Cosmetic | Content (wt %) |

| composition | 1 | 2 | 3 | 4 |
|---|---|---|---|---|
| VDR Pep 1 | 0 | 0.04 | 0 | 0 |
| VDR Pep 2 | 0 | 0 | 0.04 | 0 |
| VDR Pep 3 | 0 | 0 | 0 | 0.04 |
| Preservatives | 0.02 | 0.02 | 0.02 | 0.02 |
| Fragrance | 0.4 | 0.4 | 0.4 | 0.4 |
| Glycerin | 2 | 2 | 2 | 2 |
| Purified | Residue | Residue | Residue | Residue |
| water | | | | |

2. Twenty-one male adults with alopecia were asked to apply the same amount of each of the cosmetic compositions 1 to 4 prepared in Example 9-1 to the hair loss area every morning and evening for 10 weeks. After 10 weeks, hair condition was checked with the naked eye.

2. As a result of the experiment, the hair did not become thicker or re-grow when the control cosmetic composition 1 was applied. Meanwhile, the hair became thicker and the number of hairs increased when cosmetic compositions 2 to 4 were applied.

Herein above, the applicants have described preferred examples of the present disclosure, but such examples are only examples that implement the technical idea of the present disclosure. Any changes or modifications should be construed as falling within the scope of the present disclosure as long as the changes or modifications embody the technical idea of the present disclosure.

1-4. (canceled)

5. A cosmetic composition comprising a peptide consisting of any one amino acid sequence selected from ones represented by SEQ ID NOs: 1 to 3.

6-7. (canceled)

8. A synthetic peptide consisting of any one amino acid sequence selected from ones represented by SEQ ID NOs: 1 to 3, wherein the synthetic peptide is a chemically synthesized peptide.

9. A peptide having an activity of activating a vitamin D receptor, consisting of any one amino acid sequence selected from ones represented by SEQ ID NOs: 1 to 3.

10. A pharmaceutical composition comprising a peptide consisting of any one amino acid sequence selected from ones represented by SEQ ID NOs: 1 to 3.

11. A method for rejuvenating skin, comprising applying an effective amount of a peptide consisting of any one amino acid sequence selected from ones represented by SEQ ID NOs: 1 to 3 to the skin of a subject in need thereof.

12. A method for alleviating a skin trouble, comprising applying an effective amount of a peptide consisting of any one amino acid sequence selected from ones represented by SEQ ID NOs: 1 to 3 to the skin of a subject in need thereof.

13. The method of claim 12, wherein the skin trouble is selected from the group consisting of atopy, acne, and hair loss.


SEQUENCE LISTING (3 sequences)

SEQ ID NO: 1 - protein, 4 aa, synthetic construct
EVFG

SEQ ID NO: 2 - protein, 4 aa, synthetic construct
VIGF

SEQ ID NO: 3 - protein, 4 aa, synthetic construct
IGFA